(19) United States
(12) Patent Application Publication
(10) Pub. No.: US 2021/0093659 A1
(43) Pub. Date: Apr. 1, 2021

(54) METHOD OF BOWEL PREPARATION FOR COLONOSCOPY

(71) Applicant: James Matthew BOHNING, Jamison, PA (US)

(72) Inventor: James Matthew BOHNING, Jamison, PA (US)

(21) Appl. No.: 17/028,396

(22) Filed: Sep. 22, 2020

Related U.S. Application Data
(63) Continuation of application No. 62/906,919, filed on Sep. 27, 2019.

(51) Int. Cl. A61K 31/78; A61K 9/00; A61K 31/047; A61K 9/48

(52) U.S. Cl. CPC A61K 31/78; A61K 9/4808; A61K 31/047; A61K 9/0053

(57) ABSTRACT

Provided are methods of bowel preparation prior to a colonoscopy using superabsorbent polymer (SAP) spheres. The SAP spheres can be administered in a capsule, such as a delayed release capsule, to prevent release of the spheres from the capsule until the capsule passes into the small intestine. The SAP spheres can be administered alone with water or in combination with another liquid bowel preparation and/or laxative to clear the bowel of the subject prior to a colonoscopy procedure.

### CROSS-REFERENCE TO RELATED APPLICATIONS

This application claims priority to copending U.S. Provisional Patent Application No. 62/906,919 filed Sep. 27, 2019, which is incorporated by reference herein.

### BACKGROUND OF THE INVENTION

Colon cancer is the third leading cause of cancer deaths in the United States. Colon cancer rates have been steadily declining over the past twenty years in the United States [1] since the advent of colonoscopy, a procedure during which pre-cancerous polyps can be removed, thereby preventing polyps from becoming cancer. This process is achieved by first cleansing, or preparing, the colon of debris so that the colonoscope, a long, thin, flexible camera, may be passed from the rectum to the beginning of the colon and, when the colonic mucosa is adequately visualized, remove polyps. Poor preparation of the colon can lead to missed polyps and decrease the effectiveness of having a colonoscopy, necessitating bowel cleansing preparations (bowel preps) that are well tolerated, safe, and effective means of clearing the colon. Most bowel preps work by using osmosis to purge large volumes of water through the colon, creating a drag effect to clear the colon of debris. While bowel preps have evolved over the past thirty years, they are still mostly comprised of polyethylene glycol (PEG) alone or in combinations with sodium ascorbate, sodium sulfate, ascorbic acid, sodium chloride and potassium chloride, or combinations of sodium sulfate, potassium sulfate, magnesium sulfate [2]. These preparations can lead to dehydration, renal insufficiency and toxicity, electrolyte imbalances and associated arrythmias.

Patient satisfaction with current preps is very low due to the large volume of prep and ingested water needed, the taste of the prep, and side effects including nausea and vomiting. This leads to poor compliance with the prescribed prep, which can cause suboptimal bowel preparation and make the colonoscopy ineffective, and is also a major barrier to the willingness of patients to undergo colonoscopies. Colon cancer screening rates are currently around 58%-68% of eligible patients, leaving about 38 million adults in the United States who are not up to date with colon cancer screening [3]. Due to these factors, there is great need for an improved method of bowel preparation which avoids these pitfalls and improves patient compliance and colon cancer screening rates, which will thereby prevent more colon cancer.

### BRIEF SUMMARY OF THE INVENTION

A method for improved bowel preparation prior to colonoscopy that overcomes some of the disadvantages of prior art methods of bowel preparation is provided.

In a general aspect, provided is a method of bowel preparation in a subject in need of a colonoscopy, the method comprising administering to the subject at least one capsule comprising a plurality of super absorbent polymer (SAP) spheres, optionally in combination with water.

In some embodiments, the capsule is a delayed release capsule.

In some embodiments, the SAP spheres have an unexpanded average diameter of 0.5 mm to 8.0 mm, such as 1 mm, 2.5 mm, or 4.0 mm.

In some embodiments, the SAP spheres comprise at least one of cross-linked sodium polyacrylate, polyacrylamide, ethylene maleic anhydride copolymer, polyvinyl alcohol, cross-linked polyethylene oxide, and cross-linked carboxymethylcellulose, and preferably comprise cross-linked sodium polyacrylate.

In some embodiments, the method further comprises administering a laxative to the subject.

In some embodiments, the capsules are administered orally.

In some embodiments, a method comprises administering to the subject at least one capsule comprising a plurality of super absorbent polymer (SAP) spheres in combination with a liquid bowel preparation, optionally in combination with a laxative.

In some embodiments, the liquid bowel preparation comprises polyethylene glycol (PEG).

### BRIEF DESCRIPTION OF THE DRAWINGS

The foregoing summary, as well as the following detailed description of the invention, will be better understood when read in conjunction with the appended drawings. It should be understood that the invention is not limited to the precise embodiments shown in the drawings.

In the drawings:

FIGS. 1A and 1B show images of a delayed release capsule containing super absorbent polymer (SAP) spheres before expansion; FIG. 1A is a schematic representation of a delayed release capsule containing SAP spheres before expansion; FIG. 1B is an image of a delayed release capsule showing the size of the capsule;

FIG. 2 is a schematic representation of a colon containing expanded SAP spheres using colonic peristalsis to purge the colonic contents;

FIGS. 3A and 3B show a delayed release capsule filled with SAP spheres and the subsequently expanded spheres; FIG. 3A shows a delayed release capsule filled with eighty (80) 1 mm SAP spheres; FIG. 3B shows the volume of the expanded eighty (80) 1 mm SAP spheres;

FIGS. 4A and 4B show a delayed release capsule filled with SAP spheres and the subsequently expanded spheres; FIG. 4A shows a delayed release capsule filled with sixty (60) 2.5 mm SAP spheres; FIG. 4B shows the volume of the expanded sixty (60) 2.5 mm SAP spheres;

FIG. 5 shows growth (change in size) over time of 1 mm and 2.5 mm SAP spheres in Lactated Ringer's Solution;

FIGS. 6A and 6B show the diameter and volume of SAP spheres over time in Lactated Ringer's Solution from the study described in Example 1; FIG. 6A shows the diameter of 1 mm and 2.5 mm SAP spheres over time; FIG. 6B shows the volume of 1 mm and 2.5 mm SAP spheres over time; and

FIGS. 7A-7F show images of the clearing of simulated colonic debris from a colon model as described in Example 2; FIG. 7A shows an image of a colon model containing simulated colonic debris; FIG. 7B shows an image of a colon model, in which the parchment paper is rolled to a diameter of 5 cm; FIG. 7C shows a cross-section of the colon model shown in FIG. 7B containing simulated colonic debris; FIG. 7D shows the results of clearing of simulated colonic debris with 250 mL of water; FIG. 7E shows the results of clearing of simulated colonic debris with 1 mm SAP spheres; and FIG. 7F shows the results of clearing of simulated colonic debris with 2.5 mm SAP spheres.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the disclosure. Such discussion is not an admission that any or all of these matters form part of the prior art with respect to the disclosure.

Unless defined otherwise, all technical and scientific terms used herein have the same meaning as commonly understood to one of ordinary skill in the art to which this invention pertains. Otherwise, certain terms used herein have the meanings as set forth in the specification. All patents, published patent applications and publications cited herein are incorporated by reference as if set forth fully herein.

It must be noted that as used herein and in the appended claims, the singular forms “a,” “an,” and “the” include plural reference unless the context clearly dictates otherwise.

Unless otherwise stated, any numerical value is to be understood as being modified in all instances by the term “about.” Thus, a numerical value typically includes ±10% of the recited value. For example, the recitation of a length such as “1 mm” includes 0.9 mm and 1.1 mm. As used herein, the use of a numerical range expressly includes all possible subranges, all individual numerical values within that range, including integers within such ranges and fractions of the values unless the context clearly indicates otherwise.

In one general aspect, provided is a method of bowel preparation in a subject in need of a colonoscopy, the method comprising administering to the subject at least one capsule comprising a plurality of super absorbent polymer (SAP) spheres. Capsules comprising the SAP spheres are typically administered to a subject in combination with water.

As used herein, “subject” means any animal, preferably a mammal, most preferably a human, who will be or has been treated by a method according to an embodiment of the invention. The term “mammal” as used herein encompasses any mammal. Examples of mammals include, but are not limited to, cows, horses, sheep, pigs, cats, dogs, mice, rats, rabbits, guinea pigs, non-human primates (NHPs), such as monkeys or apes, humans, etc., more preferably a human. Preferably, the subject is in need of a surgical or medical procedure, and is more preferably in need of a colonoscopy.

As used herein, “bowel preparation,” also known as “bowel prep,” refers to the process of removing feces, food particles, and other debris from the colon (large intestine) prior to a medical or surgical procedure, such as a colonoscopy. According to embodiments described herein, bowel preparation is performed prior to a colonoscopy, a procedure in which a flexible, lighted tube equipped with a tiny TV camera, called a colonoscope, is inserted into the rectum and threaded through the colon to look for any evidence of cancer, polyps, ulcers, etc. Thus, in order for the colon to be adequately visualized, the bowel must first be prepped by removing any feces, food particles, and other debris from the colon.

As used herein, the terms “super absorbent polymer spheres” and “SAP spheres” refer to balls or spheres formed from super absorbent polymers, polymers capable of absorbing and retaining large amounts of liquid relative to the mass of the polymer. Super absorbent polymer spheres are typically formed by the reaction of a polymer and a cross-linking agent, resulting in a cross-linked polymer that is able to absorb large amounts of water by, for example, hydrogen bonding. Preferably, a super absorbent polymer sphere is capable of absorbing up to 300 times its weight in deionized water and about 50 times its weight in 0.9% saline. A super absorbent polymer sphere suitable for use in the invention is preferably non-toxic.

Super absorbent polymer spheres can be made from any super absorbent polymer known in the art in view of the present disclosure. In one embodiment, a super absorbent polymer sphere comprises a polymer made from sodium polyacrylate and a cross-linking agent, i.e., cross-linked sodium polyacrylate. Examples of suitable cross-linking agents include, but are not limited to, sodium 2-propenoate. Cross-linked sodium polyacrylate is able to absorb large amounts of water through hydrogen bonding, absorbing up to 300 times its weight in deionized water, and about 50 times its weight in 0.9% saline solution [4]. Other examples of super absorbent polymers suitable for use in the invention include, but are not limited to, polyacrylamide, ethylene maleic anhydride copolymer, polyvinyl alcohol, cross-linked polyethylene oxide, and cross-linked carboxymethylcellulose.

SAP spheres are thought to be non-toxic, with the median lethal dose (LD50) from oral administration in rat being >1600 mg/kg and in mouse being >3200 mg/kg. Reports of complications from ingestion in children have been reported, but only due to the size of the expanded SAP spheres and mechanical obstruction [5]. A 10-year retrospective human study found no pathological findings, evidence of toxicity, or poisoning from ingestion of SAP spheres [6]. A toxicity study of cross-linked sodium polyacrylate/polyvinyl alcohol copolymer in chickens showed that even a dose of 6 g of hydrated polymer had no adverse effects on 1 day old chicks [9].

According to embodiments described herein, the size of the SAP spheres does not exceed the diameter of the average-sized small intestine when the SAP spheres expand upon absorption of water or other liquid when administered to a subject. Preferably, the diameter of an SAP sphere suitable for use in the methods described herein is 0.5 mm to 8.0 mm prior to expansion (i.e., the unexpanded average diameter), such as 0.5 mm, 0.75 mm, 1 mm, 1.25 mm, 1.5 mm, 1.75 mm, 2.0 mm, 2.25 mm, 2.5 mm, 2.75 mm, 3.0 mm, 3.25 mm, 3.5 mm, 3.75 mm, 4.0 mm, 4.25 mm, 4.5 mm, 4.75 mm, 5.0 mm, 5.25 mm, 5.5 mm, 5.75 mm, 6.0 mm, 6.25 mm, 6.5 mm, 6.75 mm, 7.0 mm, 7.25 mm, 7.5 mm, 7.75 mm, or 8 mm. After expansion, the diameter of an SAP sphere is less than the average diameter of the small bowel (small intestine), which is 2.5 cm, and the expanded average diameter is preferably no larger than 5 mm to 18 mm, such as no larger than 5.0 mm 5.5 mm, 6.0 mm 6.5 mm, 7.0 mm, 7.5 mm, 8.0 mm, 8.5 mm, 9.0 mm, 9.5 mm, 10 mm, 10.5 mm, 11.0 mm, 11.5 mm, 12.0 mm, 12.5 mm, 13.0 mm, 13.5 mm, 14.0 mm, 14.5 mm, 15.0 mm, 15.5 mm, 16.0 mm, 16.5 mm, 17.0 mm, 17.5 mm, 18.0 mm.

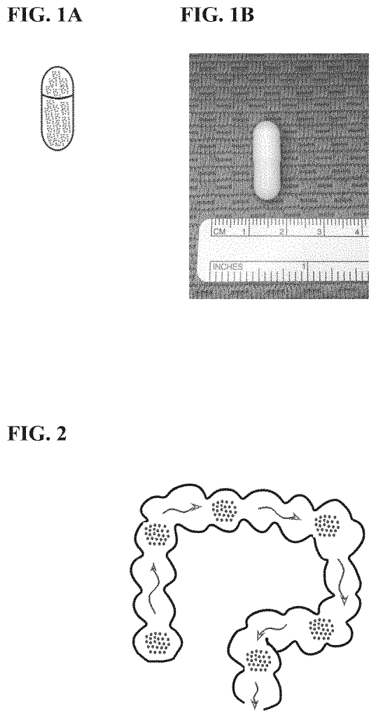

According to embodiments described herein, super absorbent polymers are administered to a subject in a capsule. Preferably, the capsule is a delayed release capsule. “Delayed release capsule” as used herein refers to a capsule or other solid dosage form that releases its contents at a time other than promptly after administration. Delayed release capsules suitable for use in the methods described herein preferably do not release SAP spheres until the capsules enter the small intestine, such that the SAP spheres are delivered in a contracted (i.e., non-expanded or unexpanded) state to the small intestine, thus preventing expansion of the SAP spheres in the stomach (FIGS. 1A and 1B). Thus, over the average small bowel (small intestine) transit time of 4-5 hours, the SAP spheres expand upon absorption of water or other liquid and then have the desired purgative effect in the colon or large intestine (FIG. 2). Preferably, the capsules used prevent expansion of the SAP spheres in the stomach to avoid satiation and other potential side effects such as bloating, nausea, and vomiting. For example, exemplary and non-limiting examples of delayed release capsules that can be used include, but are not limited to, acid resistant enteric capsules (e.g., Capsuline® Clear Vegetarian Acid Resistant Enteric Capsule); gelatin enteric coated capsules (e.g., PURECAPS® USA Gelatin Enteric Coated Capsule); and other acid resistant capsules (e.g., Capsugel® DRCaps™).

In a preferred embodiment, the capsules are administered orally. The number of capsules administered per administration and the number of administrations is not particularly limited. One or more capsules can be administered at one or more times. For example, 1 to 10, such as 1, 2, 3, 4, 5, 6, 7, 8, 9, or 10 capsules can be administered per administration, for a total of 1 to 10 administrations, such as 1, 2, 3, 4, 5, 6, 7, 8, 9, or 10 administrations. The capsules can be administered alone, with water, or with additional agents, such as other bowel prep agents (e.g., polyethylene glycol (PEG)) and/or stimulant laxatives (e.g., bisacodyl). As an illustrative and non-limiting example, 2 capsules can be administered to the subject 1, 2, or 3 times beginning 24 hours prior to the time at which the colonoscopy procedure is to be performed, optionally supplemented with a dose of PEG with water and/or a stimulant laxative (e.g., bisacodyl) 8 hours to 24 hours prior to the time at which the colonoscopy procedure is to be performed.

In another embodiment, at least one capsule comprising a plurality of super absorbent polymer (SAP) spheres is administered to a subject in combination with a liquid bowel preparation. According to embodiments of the invention, the use of SAP spheres in combination with another bowel preparation method reduces the amount of the other bowel preparation method needed in order to adequately remove feces and other debris from the colon as compared to the amount of the other bowel preparation that otherwise would be needed in the absence of the SAP spheres.

As used herein, “in combination with” refers to simultaneous administration of two or more components, such as SAP sphere filled capsules and a liquid bowel preparation. When two or more components are administered “in combination with,” they can be administered in separate compositions sequentially within a short time period, such as 24 hours, 20 hours, 16 hours, 12 hours, 8 hours, or 4 hours, or within 1 hour or less, such as 1 hour, 30 minutes, 15 minutes, 5 minutes, or 1 minute or less. For example, administration of capsule(s) comprising a plurality of SAP spheres in combination with a liquid bowel preparation can refer to administration of the two components sequentially within 1 hour or less per administration, such as 1 hour, 30 minutes, 15 minutes, 5 minutes, or 1 minute or less.

Any liquid bowel preparation known in the art in view of the present disclosure can be used in combination with the SAP spheres and capsules thereof described herein. For example, SAP spheres and capsules thereof can be administered in combination with a liquid bowel preparation comprising polyethylene glycol (PEG) and one or more electrolytes, such as sodium ascorbate, sodium sulfate, sodium bicarbonate, ascorbic acid, magnesium sulfate, sodium chloride, potassium chloride, etc. In one embodiment, the liquid bowel preparation comprises PEG in combination with potassium chloride, sodium bicarbonate, and sodium chloride (e.g., NuLYTELY®). In another embodiment, the liquid bowel preparation comprises PEG in combination with sodium sulfate, sodium bicarbonate, sodium chloride, and potassium chloride (e.g., GoLYTELY®). In yet another embodiment, the liquid bowel preparation comprises PEG in combination with sodium ascorbate, sodium sulfate, ascorbic acid, sodium chloride and potassium chloride (e.g., Plenvu). In other embodiments, the liquid bowel preparation comprises a mixture of sodium picosulfate, magnesium oxide, and anhydrous citric acid (e.g., CLENPIQ®). In yet other embodiments, the liquid bowel preparation comprises a mixture sodium sulfate, potassium sulfate, and magnesium sulfate (e.g., SUPREP®).

It is also possible to administer a laxative in combination with the SAP spheres or capsules thereof. Thus, in some embodiments, the methods described herein further comprise administration of a laxative in combination with the SAP spheres or capsules thereof, optionally in combination with a liquid bowel preparation. Examples of laxatives suitable for use with the invention include, but are not limited to, bisacodyl (e.g., Dulcolax®).

A bowel preparation for colonoscopy using super absorbent polymer (SAP) spheres delivered in a delayed release capsule as described herein provides an improved bowel prep that is tasteless and odorless with less ingested volume, delivering an acceptable bowel preparation without the same side effects of current bowel preps. Without wishing to be bound by any theories, given their physical properties of maintaining shape, though compressible, it is believed that expanded SAP spheres represent an advantageous substrate to purge colonic contents using the peristalsis of the colon to propel the water containing spheres through the colon. In contrast, currently used liquid bowel preps require large amounts of volume to attain the desired drag effect and effectively remove all debris from the colon. Given the physical nature and increased friction of a solid over a liquid, the use of SAP spheres provides for a more efficient use of the amount of water ingested, and therefore requires ingestion of much less water (e.g., analogous to using a sponge to clean a dish rather than rinsing alone). The side effects of nausea, vomiting and poor taste due to electrolytes contained in current bowel preparations would also be eliminated, which is a further advantage of the methods described herein.

### EXAMPLES

The following examples of the application are to further illustrate the nature of the application. It should be understood that the following examples do not limit the application and the scope of the application is to be determined by the appended claims.

### Example 1: Preparation of Capsules Containing Super Absorbent Polymer (SAP) Spheres and Expansion of SAP Spheres

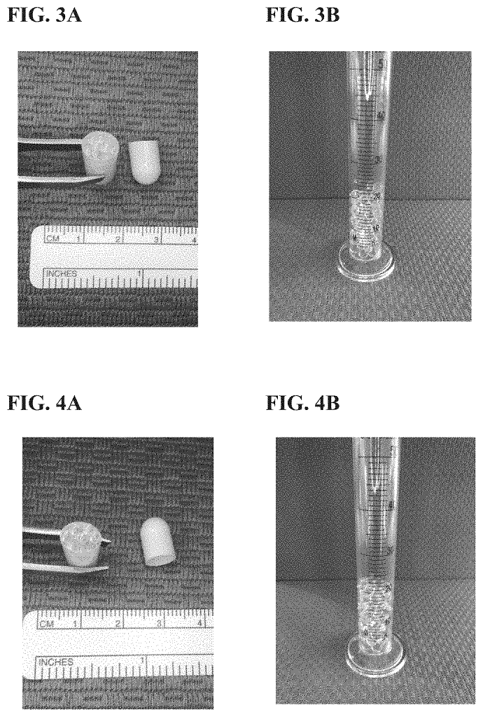

Acid resistant gelatin based enteric coated delayed release capsules (PURECAPS® USA Gelatin Enteric Coated Capsules, size “0” (0.68 mL)) were filled with either eighty (80) 1 mm SAP spheres or sixty (60) 2.5 mm SAP spheres (FIGS. 3A and 4A). The SAP spheres comprised 2-propenoic acid polymer crosslinked with sodium 2-propenoate (cross-linked sodium polyacrylate; M2 Polymer Technologies, Inc.).

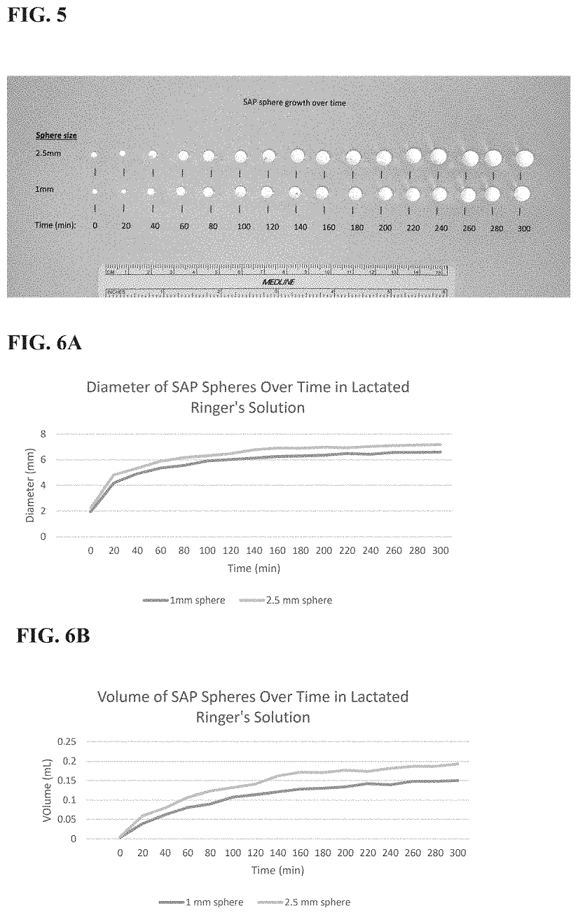

The SAP spheres were placed in physiological saline solution (Lactated Ringer's solution, pH 6.2, Na⁺ 130 milliequivalents per liter (mEq/L), K⁺4 mEq/L, Cl²⁺ 110 mEq/L, Ca²⁺3 mEq/L, Lactate 28 mEq/L) and allowed to expand for five (5) hours. The maximal expansion in 5 hours of the 1 mm SAP spheres was found to be a diameter of 6.59 mm (volume 0.150 mL, 37.5× the original volume); and the maximal expansion in 5 hours of the 2.5 mm SAP spheres was found to be a diameter of 7.17 mm (volume 0.193 mL, 32.2× the original volume). See Tables 1 and 2; and FIGS. 5, 6A, and 6B.

TABLE 1
Diameter and Volume of 1 mm SAP Spheres
over time in Lactated Ringer's Solution

| Time (min) | Diameter (mm) | Volume (ml) |
|---|---|---|
| 0 | 1.94 | 0.004 |
| 20 | 4.19 | 0.039 |
| 40 | 4.91 | 0.062 |
| 60 | 5.36 | 0.081 |
| 80 | 5.56 | 0.090 |
| 100 | 5.91 | 0.108 |
| 120 | 6.02 | 0.114 |
| 140 | 6.14 | 0.121 |
| 160 | 6.25 | 0.128 |
| 180 | 6.29 | 0.130 |
| 200 | 6.35 | 0.134 |
| 220 | 6.48 | 0.142 |
| 240 | 6.42 | 0.139 |
| 260 | 6.57 | 0.148 |
| 280 | 6.56 | 0.148 |
| 300 | 6.59 | 0.150 |

TABLE 2
Diameter and Volume of 2.5 mm SAP Spheres
over time in Lactated Ringer's Solution

| Time (min) | Diameter (mm) | Volume (ml) |
|---|---|---|
| 0 | 2.23 | 0.006 |
| 20 | 4.82 | 0.059 |
| 40 | 5.35 | 0.080 |
| 60 | 5.89 | 0.107 |
| 80 | 6.17 | 0.123 |
| 100 | 6.31 | 0.132 |
| 120 | 6.46 | 0.141 |
| 140 | 6.77 | 0.162 |
| 160 | 6.90 | 0.172 |
| 180 | 6.89 | 0.171 |
| 200 | 6.97 | 0.177 |
| 220 | 6.93 | 0.174 |
| 240 | 7.03 | 0.182 |
| 260 | 7.10 | 0.187 |
| 280 | 7.14 | 0.191 |
| 300 | 7.17 | 0.193 |

After 24 hours of absorbing water in the physiological Lactated Ringer's solution, the maximum diameter reached for the 1 mm SAP spheres was 6.64 mm, and the maximum diameter reached for the 2.5 mm SAP spheres was 7.36 mm. In only 5 hours of absorption, the 1 mm SAP spheres reached 99.2% of the maximum diameter observed and the 2.5 mm SAP spheres reached 97.4% of the maximum diameter observed. The greatest rate of expansion was found in the first 20-minute period with, on average, a decreasing rate of expansion every interval thereafter. Though the increase in diameter size was a linear relationship from the 1 mm to 2.5 mm sphere, expansion volume was related to the cube of the radius (V=4/3πr³). Therefore, the volume of the 2.5 mm spheres increased more than the volume of the 1 mm spheres (δ 0.187 ml vs. δ 0.146 ml). Given that more 1 mm spheres could fit into a single capsule (80 spheres) than 2.5 mm spheres (60 spheres), the overall volume combined of the expanded 1 mm spheres was greater (12 mL vs. 11.58 mL).

These results demonstrate that the SAP spheres do not expand to a size larger than the average small bowel diameter of 2.5 cm over a period of 24 hours.

### Example 2: Clearing of Simulated Colonic Debris from Colon Model

Objective clearing of simulated colonic debris (mixture of ground bran flakes, peanut butter, water) by eighty (80) 1 mm SAP expanded spheres, sixty (60) 2.5 mm SAP expanded spheres, and 250 mL of water from a model colon with simulated peristalsis was evaluated. The model colon used was parchment paper rolled to a diameter of 5 cm and the simulated peristalsis was a roller moved across the model colon. More specifically, expanded, loose 1 mm or 2.5 mm SAP spheres were placed at the beginning of the model colon. The roller was moved once across the colon model to simulate peristalsis. For the water experiment, 250 mL of water was injected into the beginning of the model colon with a piston syringe, and the roller was moved once across the colon model to simulate peristalsis.

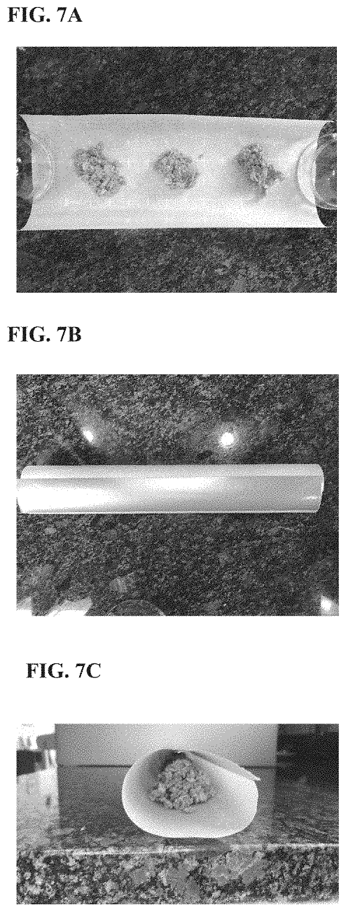

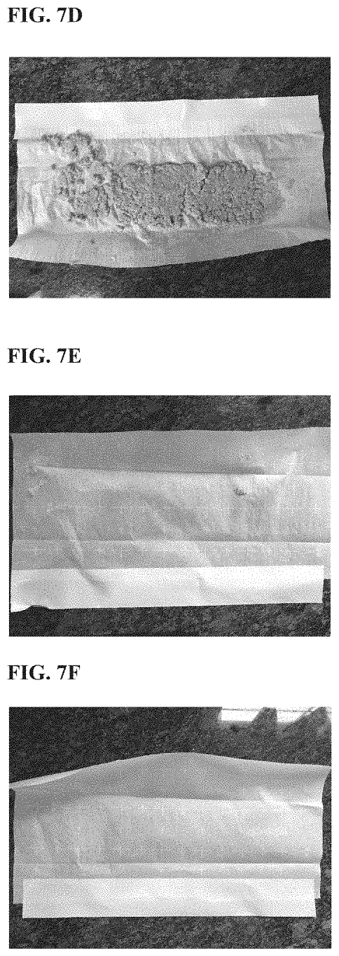

The results demonstrated that simulated debris removal was far superior by both the expanded 1 mm and expanded 2.5 mm SAP sphere preparations (FIGS. 7E and 7F, respectively) as compared to removal with 250 mL of water (FIG. 7D).

### REFERENCES

1. National Colorectal Cancer Roundtable, https://nccrt.org/data-progress/
2. Adamcewicz, A, Friedenberg, F, et al. “Mechanism of Action and Toxicities of Purgatives Used for Colonoscopy Preparation”, Expert Opin Drug Metab Toxicol, 2011 January; 7(1) p 89-101
3. National Colorectal Cancer Roundtable, https://nccrt.org/data-progress/
4. Wikipedia, https://en.wikipedia.org/wiki/Superabsorbent_polymer
5. Mohamed, A, Hassan, M, et al, “Bowel Obstruction by Ingestion of Superabsorbent Polymer Balls”, Journal of Ped Surg Case Reports, 2019 February, Vol. 41 p 27-29
6. Mehmetoglu, F, “A Retrospective 10-Year Analysis of Water Absorbent Bead Ingestion in Children”, Emerg Med International, Vol. 2018
7. U.S. Pat. No. 10,098,907
8. U.S. Patent Application Publication No. 2002/0146386
9. Haselbach, J, et al. “Single-dose oral toxicity study of a cross-linked sodium polyacrylate/polyvinyl alcohol copolymer in chickens (Gallus domesticus)”, Regul Toxicol Pharmacol, 2000

I claim:

1. A method of bowel preparation in a subject in need of colonoscopy, the method comprising administering to the subject at least one capsule comprising a plurality of super absorbent polymer (SAP) spheres.

2. The method of claim 1, wherein the capsule is a delayed release capsule.

3. The method of claim 1, wherein the SAP spheres have an unexpanded average diameter of 0.5 mm to 8.0 mm.

4. The method of claim 3, wherein the SAP spheres have an unexpanded average diameter of 1 mm.

5. The method of claim 3, wherein the SAP spheres have an unexpanded average diameter of 2.5 mm.

6. The method of claim 3, wherein the SAP spheres have an unexpanded average diameter of 4.0 mm.

7. The method of claim 1, wherein the SAP spheres comprise at least one of cross-linked sodium polyacrylate, polyacrylamide, ethylene maleic anhydride copolymer, polyvinyl alcohol, cross-linked polyethylene oxide, and cross-linked carboxymethylcellulose.

8. The method of claim 7, wherein the SAP spheres comprise cross-linked sodium polyacrylate.

9. The method of claim 1, further comprising administering a laxative to the subject.

10. The method of claim 1, wherein the at least one capsule is administered orally.

11. A method of bowel preparation in a subject in need of a colonoscopy, the method comprising administering to the subject at least one capsule comprising a plurality of super absorbent polymer (SAP) spheres in combination with a liquid bowel preparation, optionally in combination with a laxative.

12. The method of claim 11, wherein the liquid bowel preparation comprises polyethylene glycol (PEG).

13. The method of claim 11, wherein the capsule is a delayed release capsule.

14. The method of claim 11, wherein the SAP spheres have an unexpanded average diameter of 0.5 mm to 8.0 mm.

15. The method of claim 14, wherein the SAP spheres have an unexpanded average diameter of 1 mm.

16. The method of claim 14, wherein the SAP spheres have an unexpanded average diameter of 2.5 mm.

17. The method of claim 14, wherein the SAP spheres have an unexpanded average diameter of 4.0 mm.

18. The method of claim 11, wherein the SAP spheres comprise at least one of cross-linked sodium polyacrylate, polyacrylamide, ethylene maleic anhydride copolymer, polyvinyl alcohol, cross-linked polyethylene oxide, and cross-linked carboxymethylcellulose.

19. The method of claim 18, wherein the SAP spheres comprise cross-linked sodium polyacrylate.

20. The method of claim 11, wherein the at least one capsule is administered orally.
